(19) United States
(12) Patent Application Publication
(10) Pub. No.: US 2019/0366154 A1
(43) Pub. Date: Dec. 5, 2019

(54) PHYSICAL ACTIVITY TRAINING ASSISTANT

(71) Applicant: Microsoft Technology Licensing, LLC, Redmond, WA (US)

(72) Inventor: David M. CALLAGHAN, Redmond, WA (US)

(21) Appl. No.: 15/995,100

(22) Filed: May 31, 2018

(51) Int. Cl. A63B 24/00; A63B 21/072; A61B 5/11

(52) U.S. Cl. CPC A63B 24/0062; A63B 21/078; A61B 5/1118; A63B 21/0724

(57) ABSTRACT

The devices, systems, and methods described herein enable an automatic training assistant for physical activity by receiving sensor data representing an actual path of motion of a user during a physical activity, comparing the received sensor data to an identified activity model that includes an expected path of motion corresponding to the user's physiology, identifying a deviation from the identified activity model based on the comparison, generating a suggestion based on the identified deviation to remediate the identified deviation, and presenting the generated suggestion to the user. The automatic training assistant enables activity detection frameworks that automatically identify weaknesses of the user's performance of a particular physical activity, automatically generate suggestions to remediate such weaknesses, and optionally track the effectiveness of the suggestions.

### BACKGROUND

Contemporary fitness oriented wearable devices such as mobile phones, smart watches, fitness trackers, and the like are capable of automatically detecting various physical activities performed by a user. For example, wearable devices may detect cardiovascular exercises such as walking, running, biking, and swimming, as well as strength training exercises such as squats, bench presses, sit ups, and push-ups. The more advanced devices can detect the exercises with less information as an input or hinting from the user on the exercise performed or the number of repetitions, duration etc. Automatically detecting the activity performed by the user may be less intrusive to the user's activity by enabling the user to spend less time fidgeting with the device and more time focused on training. The wearable devices may automatically track various statistics related to the activity, such as steps, repetitions, distance, pace, speed, elevation, route, and the like. But, automatically tracking such statistics may be of limited use to a user for improving the user's form and ability on a particular physical activity. As a result, many physical activities still require a personal trainer. One advantage of a personal trainer is ensuring the activity is performed with proper form to prevent injury and maximize effectiveness of the training.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

A computerized method comprises receiving, at an electronic device, sensor data representing an actual path of motion of a user during a physical activity, comparing the received sensor data to an identified activity model that includes an expected path of motion corresponding to the user's physiology, identifying a deviation from the identified activity model based on the comparison, generating a suggestion based on the identified deviation to remediate the identified deviation, and presenting the generated suggestion to the user.

### BRIEF DESCRIPTION OF THE DRAWINGS

The present description will be better understood from the following detailed description read in light of the accompanying drawings, wherein:

FIG. 1 is an exemplary block diagram illustrating an electronic device including an automatic training assistant module according to an embodiment.

FIG. 2A is a diagram illustrating an exemplary physical activity performed by a user.

FIG. 2B is a diagram illustrating a deviation from a path of motion of the physical activity shown in FIG. 2A.

FIG. 3 is an exemplary flow chart illustrating a method of automatic physical training using an electronic device according to an embodiment.

FIG. 4 is an exemplary flow chart illustrating a method of automatic physical training using an electronic device according to an embodiment.

FIG. 5 illustrates an electronic device according to an embodiment as a functional block diagram.

Corresponding reference characters indicate corresponding parts throughout the drawings.

### DETAILED DESCRIPTION

Referring to the figures, the devices, systems, and methods described herein enable an automatic training assistant for physical activity. The devices, systems, and methods enable receiving sensor data representing an actual path of motion of a user during a physical activity, comparing the received sensor data to an identified activity model that includes an expected path of motion corresponding to the user's physiology, identifying a deviation from the identified activity model based on the comparison, generating a suggestion based on the identified deviation to remediate the identified deviation, and presenting the generated suggestion to the user. The automatic training assistant enables activity detection frameworks that automatically identify weaknesses of the user's performance of a particular physical activity and automatically generate suggestions (e.g., supplemental activities) to remediate such weaknesses.

In some embodiments, the user's path of motion for specific body parts that are not wearing sensors may be inferred from sensors data on adjacent or related body parts. A fitness watch worn on a left wrist while the user grasps a straight barbell and performs a bicep curl exercise can observe the path of motion of the bar as well as any twisting motion and acceleration and deceleration of the wrist to infer the bar is not traveling horizontally up and down in an ideal arc during a bicep curl repetition.

In other embodiments, the user's path of motion can be combined from a plurality of sensors such as a fitness wearable like a watch worn on one arm or leg and a smartphone carried on the body in a pocket or worn on an adjacent arm or leg. Additional sensors can be added to the fitness equipment and users clothing to form an integrated network of data gathering during the fitness activities.

The devices, systems, and methods described herein execute operations in an unconventional manner to enable improved performance of a variety of physical activities without the use of a human personal trainer.

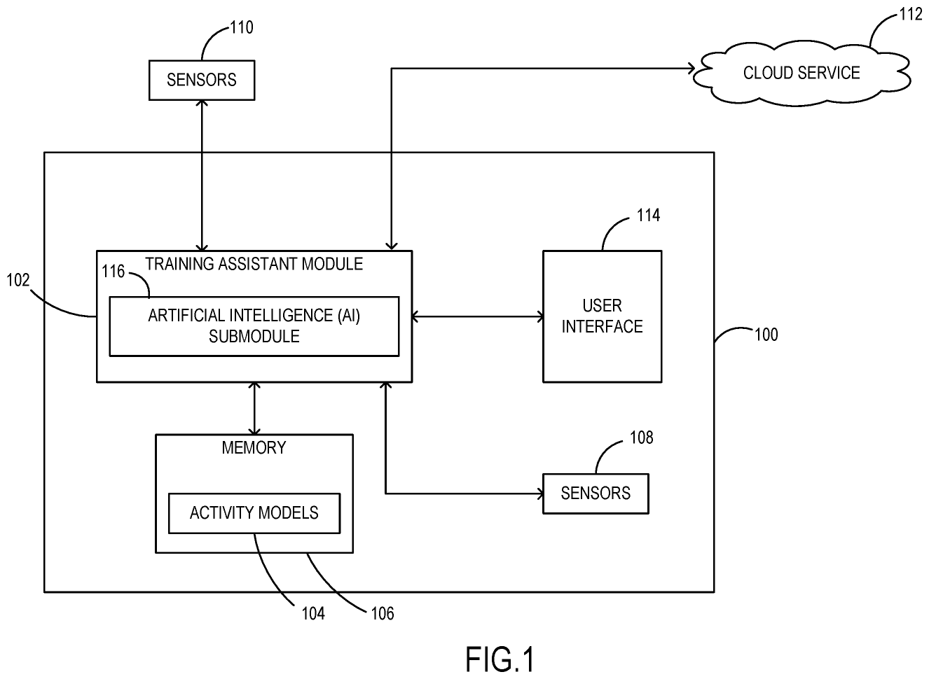

Referring to FIG. 1, an exemplary block diagram illustrates an electronic device 100 including an automatic training assistant module 102 according to an embodiment. As described in more detail herein, the training assistant module 102 compares sensor data that represents an actual path of motion of a user during a physical activity with an expected path of motion of an activity model 104 that corresponds to the user's physiology and to a particular physical activity. Based on the comparison, the training assistant module 102 identifies one or more deviations of the actual path of motion from the expected path of motion and generates one or more suggestions based on the identified deviation to remediate the deviation.

The training assistant module 102 comprises software stored in memory and executed on a processor in some cases. In some examples, the training assistant module 102 is executed on an Field-programmable Gate Array (FPGA) or a dedicated chip. For example, the functionality of the training assistant module 102 may be implemented, in whole or in part, by one or more hardware logic components. For example, and without limitation, illustrative types of hardware logic components that can be used include FPGAs, Application-specific Integrated Circuits (ASICs), Application-specific Standard Products (ASSPs), System-on-a-chip systems (SOCs), Complex Programmable Logic Devices (CPLDs), Graphics Processing Units (GPUs), and/or the like.

The electronic device 100 represents any device executing instructions (e.g., as application programs/software, operating system functionality, or both) to implement the operations and functionality associated with the electronic device 100. The electronic device may include a mobile electronic device or any other portable device. In some examples, the mobile electronic device includes a mobile telephone, laptop, tablet, computing pad, netbook, gaming device, personal digital assistant, portable media player, smart watch, a wearable device, and/or the like. For example, the electronic device 100 may be, or include, a wearable device having a wearable and/or accessory form factor, such as, but not limited to, a smart watch, clothing, fabric, a mobile telephone, a fitness tracker, a portable media player, a heart rate monitor, a blood pressure sensor, a camera, a headset, glasses, earphones, and/or the like. The electronic device 100 may also be embodied in less portable devices such as desktop personal computers, servers, kiosks, tabletop devices, media players, industrial control devices, gaming consoles, wireless charging stations, and electric automobile charging stations. In some examples, the electronic device 100 is incorporated into a cloud service. Additionally, the electronic device may represent a group of processing units or other computing devices.

The electronic device 100 includes a memory 106 that stores the activity models 104. Each activity model 104 is a model of a particular physical activity that may be performed by the user. Any physical activity may be modeled by the activity models 104, such as, but are not limited to, walking, running, biking, climbing stairs, using an elliptical machine, swimming, other cardiovascular exercises, squats, bench presses, sit ups, push-ups, clean and jerks, dead lifts, pulling a sled, other strength building exercises, swinging a racket, swinging a golf club, swinging a hockey stick, swinging a lacrosse stick, rowing, rock climbing, climbing an artificial structure (e.g., a building, an artificial rock wall, etc.), climbing a natural structure (e.g., a tree, a rock wall, etc.), and/or the like. Additional activity models may be stored and retrieved from a cloud service 112.

Alternatively, in other embodiments the electronic device 100 may temporarily store and transmit the activity sensor data to the cloud service 112 for analysis. The cloud service 112 may determine the physical activity from models similar to the activity models 104, such as, but are not limited to, walking, running, biking, climbing stairs, using an elliptical machine, swimming, other cardiovascular exercises, squats, bench presses, sit ups, push-ups, clean and jerks, dead lifts, pulling a sled, other strength building exercises, swinging a racket, swinging a golf club, swinging a hockey stick, swinging a lacrosse stick, rowing, rock climbing, climbing an artificial structure (e.g., a building, an artificial rock wall, etc.), climbing a natural structure (e.g., a tree, a rock wall, etc.), and/or the like.

Each activity model 104 models the corresponding physical activity by including data or data models which represent an expected path of motion that represents the path of motion taken by an individual performing the corresponding physical activity. As used herein, the phrase “path of motion” includes both the physical path taken by the user (e.g., arc/lines of motion, route, etc.) and the speed that the user moves along the physical path. The expected path of motion of each activity model 104 may be the form (e.g., arc/lines) of the motion taken by the individual performing the corresponding physical activity, or may be the route taken by the individual performing the corresponding physical activity. For example, the expected path of motion of a particular activity model 104 may be the form of data points connecting the motion made by an individual performing a bench press, a squat, a push up, a sit up, a swing, a rowing motion, and/or the like. Moreover, and for example, the expected path of motion of a particular activity model 104 may be the route taken while an individual is running, swimming, climbing, and/or the like.

The expected path of motion of each activity model 104 is segmented into a plurality of time segments to construct the path of motion of the corresponding physical activity. The time segments represent different portions (e.g., sections, segments, etc.) of the expected path of motion of the corresponding activity model 104. For example, one or more time segments of an expected path of motion corresponding to the form of a swinging motion may represent the back swing, while one or more other time segments represent the contact portion of the swing, and one or more other time segments represent the follow through of the swinging motion. Moreover, and for example, the time segments of an expected path of motion representing a running route may represent various different segments of the running route such as a hill segment, a beginning leg, a middle leg, or a finishing leg of the route.

Each expected path of motion may be segmented into any number of time segments, each of which may have any time value (e.g., may be any interval), such as, but not limited to, 1 microsecond (us), 5 milliseconds (ms), 10 ms, 1 second, and/or the like. The wide variety of time segment analysis exists because the data sampling rate for elite athletes can be on the order of magnitude of microseconds at critical moments while performing an exercise whereas a novice may be performing an exercise where the high frequency of data sampling which represent numerous but very short time segments with high accuracy of position through data sampling is not necessary. The data sampling rate for exercise segments of interest should follow typical engineering best practices. One sampling recommendation is the Nyquist—Shannon sampling theorem which establishes a sample rate that is sufficient to capture the information from a continuous time signal. The time segments of the expected path of motion of each activity model 104 may or may not have the same time value as the other time segments of the expected path of motion of the same activity model 104. For example, all of the time segments of the expected path of motion of a particular activity model 104 may have the same value. Moreover, one or more of the time segments of the expected path of motion of a particular activity model 104 may have a different value as compared to other time segments of the expected path of motion of the same activity model 104. For example, the time segment(s) representing the back swing of a swinging motion may have a different time value (sample rate) as compared to the time segment(s) that represent the contact portion and/or the follow through of the swinging motion.

The expected path of motion of each activity model 104 corresponds to, or may be adjusted to, the user's physiology. For example, individuals with different physiological characteristics may have different expected paths of motion for a given physical activity. For example, individuals with longer arms may have a different bench press form as compared to individuals with shorter arms. Accordingly, matching the expected path of motion of the activity models 104 to the user's physiology facilitates providing a more accurate comparison of the user's actual path of motion to the expected path of motion. Activity models 104 that best match the user's physiology may be selected from a variety of template activity models that are constructed from different physiological characteristics. In some examples, activity models 104 are selected from a variety of template expert activity models that are constructed from experts in the corresponding physical activity.

The expected path of motion of each activity model 104 corresponds to, or may be adjusted to, the user's level of skill with the activity. The activity model 104 can model a path of motion that allows for more error for a novice individual vs. a more stringent enforcement of path of motion for an advanced individual. For example, expected speed information may also be modeled which relates to the various acceleration and decelerations the individual is expected to perform along the physical path of motion for an exercise. The data models can allow for slight variances from the ideal path of motion. The activity models can be designed to allow for larger or smaller amounts of error in the activity path throughout the motion based on the experience level (e.g. novice vs. advanced) of the individual performing the exercise. A novice bench press activity model may only track the bar speed down and up, such as four seconds down to the chest and two seconds up to locked out arms, while the advanced individual tracking may look for a smooth deceleration down in under four seconds with a slight one to two second pause at the bottom before the individual presses the bar back up with a smooth tapering off acceleration from to the bar touching the chest until the arms are locked out in any time less than two seconds.

In addition or alternatively to selecting the activity models 104 from template activity models, the training assistant module 102 may include a model training functionality wherein the user's actual path of motion is observed (e.g., by the sensors 108 and/or 110 described herein) while the user performs a particular physical activity to establish a baseline path of motion of the user for the physical activity. The activity may be observed from sensor data and/or image capture devices such as cameras or other optical sensors along with software that identifies objects in the images as well as the user's skeletal motions along with the option to track the machine exercise equipment when involved. The training assistant module 102 reads the sensor data that represents the baseline path of motion and constructs the expected path of motion (or adjusts an existing path of motion) of the corresponding activity model 104. The training assistant module 102 may have partial or complete sets of data entered by the user into the user interface 114, or retrieved from the user profile in the cloud service 112, or shared with the device from a configuration file, or from other sensors 110 that identify the relevant user skeletal structure such as their height, weight, arm span, arm length, leg length, etc. that can be factored into determining the preferred path for various exercises.

In some examples, the activity models 104 are created by the training assistant module 102. In addition, or alternatively, the activity models 104 are created in a cloud service (e.g., the cloud service 112 described herein) that is communicatively coupled to the training assistant module 102.

It should be appreciated that this subject invention includes partitioning the implementation where the activity models 104, an artificial intelligence (AI) sub-module 116, the training assistant module 102, and the user interface 114 can be distributed between the device 100 and the cloud service 112. Sensor data could be obtained with optional additional environmental hints such as GPS or location information (e.g., at a bench press station, ¼ mile oval track, etc.) that is presented on the user interface 114 and/or is accessible in the cloud service 112 using an application or web browser on the device 100 or alternative personal computer, tablet, etc.

It should be appreciated the user interface 114 can include graphs displaying the individual path or performance information as compared to the ideal performance expected for the skill or athletic level of the individual (novice, intermediate, advanced, elite, etc.) The display may include video or animation of a character showing how the user preformed the exercise compared to the ideal model, so the individual can discern what should have been performed.

The user interface 114 can include features and configurable options such as, but not limited to, displaying an image indicating correct vs. incorrect repetitions, vibrating or making sounds and/or other alerts, and/or the like to inform the user when the user perform an exercise correctly vs. incorrectly while the user is going through a set of repetition(s) and/or during duration of the exercise (e.g., on pace going too fast or too slow, correct path of motion, moving the weight or body part(s) too fast or too slow, heart rate too high or too low, body angle leaning too much, etc.) as appropriate for informing the user of the correct vs. incorrect performance of the activity.

Internal sensors 108 and/or external sensors 110 are provided to observe the user performing a variety of physical activities. For example, the sensors 108 and 110 record sensor data representing the actual path of motion of the user during physical activities. As with the expected paths of motion of the activity models 104, the actual paths of motion recorded by the sensors 108 and/or 110 are each segmented into a plurality of time segments. As described herein, the sensor data recorded by the sensors 108 and 110 for a particular physical activity is compared by the expected path of motion of the corresponding activity model 104. Each sensor 108 and 110 may be any type of sensor that facilitates recording an actual path of motion of the user during a physical activity, such as, but not limited to, an accelerometer, a strain gauge, a camera for capturing still images, burst images, and/or video, a pedometer or other step counter, a heart rate monitor, a blood pressure sensor, a proximity sensor, a timer, a gyroscope, and/or the like. The sensors can also include image capture systems and software that perform image recognition functions that help determine the activity the user is performing. Other sensors may be image capture devices and software that can perform skeletal tracking such as what is found in Microsoft® Kinect or similar products. It should be appreciated that the subject invention also includes any combination of the aforementioned sensors working alone or in conjunction with other sensors wherein the data is analyzed in subsequent stages or compared against other sensor data to identify the exercise performed and, where relevant, the path of the resistance or athlete's limbs, etc.

It should be appreciated that any given exercise can have one or multiple acceptable and/or even ideal paths of motion depending on the user's technique. For example, the bench press can be brought lower on the sternum or higher up closer to the collar bone depending on the user's grip width (for example) and the corresponding activity model 104 can detect from the sensors 108 and/or 110 to identify if/when correction should be suggested. The activity models 104 can even be designed to allow for some variations between repetitions and/or sets, etc. Other activity models 104 can include complex intermixing in a set, for example a classic dumbbell curl where the users grip is primarily in a horizontal position and in the next repetition the grip is in a vertical position (often called a “hammerhead curl”), without flagging these as improper paths of motion. The AI sub-module 116 can help make these recommendations of proper vs. improper paths of motion based on the user's patterns of behavior, the workout of the day (WOD) posted for that training session online, via spoken commands heard by the device 100, sent to the device 100, entered in the user interface 114, and/or online in the cloud service 112, etc.

Optionally, the electronic device 100 includes the internal sensors 108, which are components of the electronic device 100 that are communicatively coupled to the training assistant module 102 for transmitting the recorded sensor data thereto. For example, in embodiments wherein the electronic device 100 is a mobile telephone or a portable media player, the electronic device 100 may include a camera that films the user while the user is performing the physical activity. In addition or alternatively to the internal sensors 108, the training assistant module 102 is communicatively coupled to the external sensors 110 for receiving recorded sensor data therefrom. The external sensors 110 may be mounted on or proximate to equipment used by the user during the physical activity. For example, an external sensor 110 may be mounted on or proximate to a bar bell or other weight carrying device, a regular bicycle, a stationary bicycle, an elliptical machine, and/or the like. In some examples, one or more external sensors 110 is carried by the user (e.g., a strain gauge and/or accelerometer contained or integrated in clothing or another wearable fabric, a heart rate monitor and/or blood pressure sensor worn by the user, etc.). The electronic device 100 may include sensors 110 located on the exercise equipment and paired with the device 100 (e.g., over Bluetooth, 802.11 Wi-Fi, etc.) for receiving data from the exercise equipment such as, but not limited to, the distance traveled detected by a treadmill, repetitions counted by an exercise machine, etc. The data may be shared directly with the device 100 and/or shared via the cloud service 112 where the exercise equipment posts the data to a cloud provider that is shared with cloud service 112 and/or leverages the communication network capabilities of device 100 to reach the cloud service 112.

The training assistant module 102 is communicatively coupled to the sensors 108 and 110 for receiving the recorded sensor data that represents the actual path of motion of the user during a physical activity. In some examples, the training assistant module 102 automatically detects the particular physical activity that is being performed by the user. For example, the training assistant module 102 reads the sensor data that represents the actual path of motion of the user for the physical activity being performed. The training assistant module 102 compares the sensor data to the activity models 104 and identifies the activity model 104 that best corresponds to the physical activity that is being performed by the user. As an alternative to such automatic detection, the user may manually input the physical activity the user intends to perform using the user interface 114 (described herein) to enable the training assistant module 102 to identify the activity model 104 that corresponds to the physical activity.

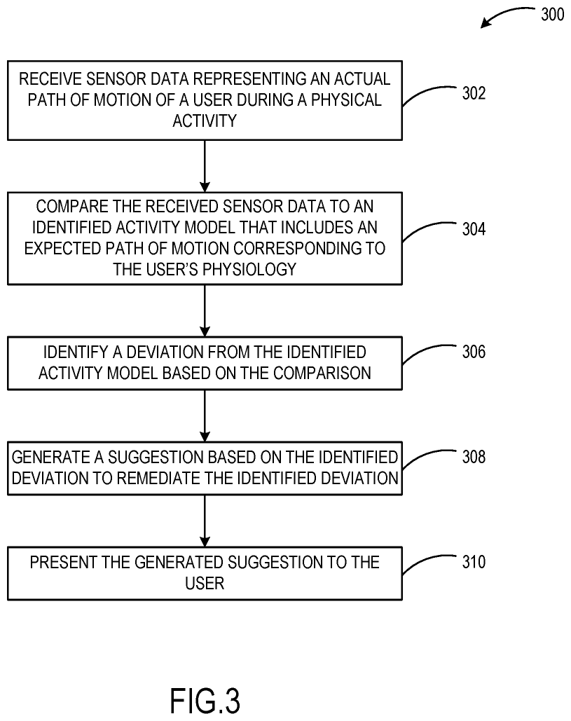

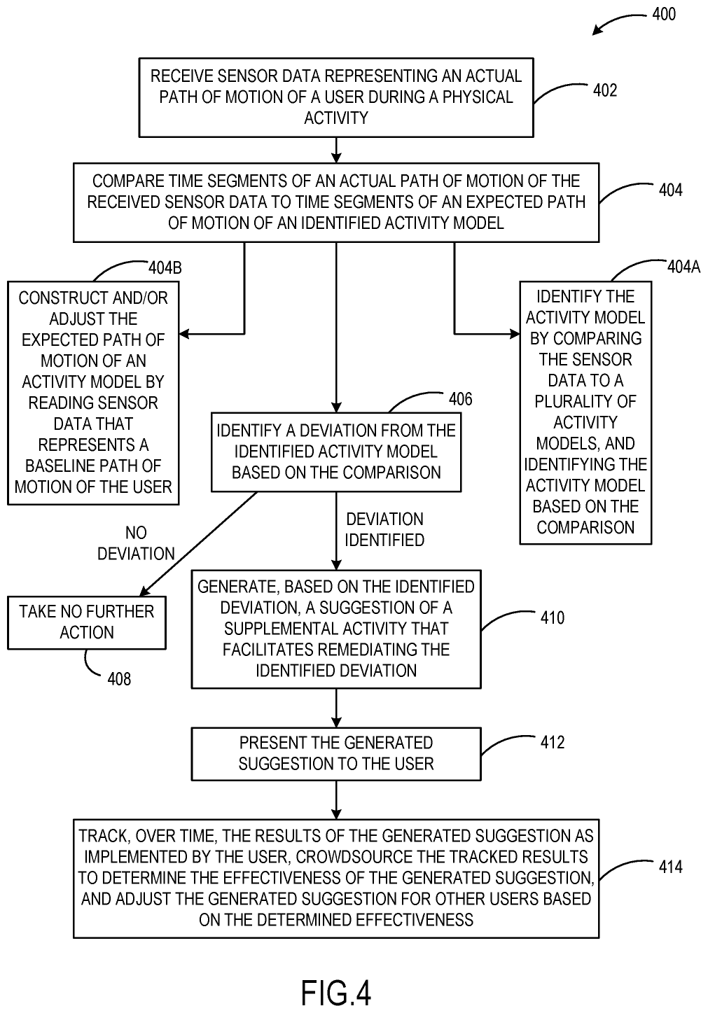

The training assistant module 102 is arranged to execute the methods described herein with respect to FIGS. 3 and 4 to identify areas of weakness of the user's performance of a physical activity and recommend accessory training activities, or form adjustments, to fix the weakness. As used herein, a weakness of the user's performance of a physical activity may include, but is not limited to, an improper or non-ideal form of the path of motion of the particular physical activity, a performance or strength deficiency impairing the user's ability to perform the physical activity (e.g., where a user is failing during a physical activity), and/or the like. The training assistant module 102 identifies the areas of weakness by comparing the received sensor data that represents the actual path of motion of the user to the identified activity model 104 and identifying a deviation from the identified activity model based on the comparison. For example, the training assistant module 102 compares each of the time segments of the actual path of motion of the received sensor data to the corresponding time segments of the expected path of motion of the identified activity model to identify any deviations therebetween.

The deviations indicate the areas of weakness of the user's performance of the physical activity. For example, a deviation between corresponding time segments of the actual and expected path of motion may indicate an improper or non-ideal form of the actual path of motion as compared to the proper form for the particular physical activity. In one specific example, a deviation of corresponding time segments near the end of a squat exercise may indicate that the user is not squatting deep enough. The user may also simply be performing the exercise in a safe and valid path but one that is not best aligned with their morphology, such as leg length versus torso length and the torso or back angle, knee position over the foot when doing squats, etc. The training assistant module 102 may include a Kinanthropometry module which optimizes the exercise recommendations, paths of motion and recovery recommendations based on the user's size, shape, proportion, composition, maturation, gross function, nutrition, recovery, rest, etc., and select various paths for the exercise and changes to the exercise routines etc. The Kinanthropometry data of the training assistant module 102 can reside on the device 100 and/or in the cloud 120, and move between the device 100 and the cloud 120 as the data is accessed and used to make or select the preferred paths, or choose from among several viable options and even allow for the user to alternate between various good, better and best paths with or without implying the user was improperly performing the exercises. For example, the training routine may include several sets of front squats and then alternate with back squats to improve the quad muscles without implying the user was performing the back squat incorrectly. The squat training assistant in this example may alternate between high bar and low bar, or choose one over the other based on the user's morphology. The training assistant module 102 component in this example learns from the databases of the users performing the exercises to identify how to get maximum benefits, comes up with a new classification system that defines new body types when needed, and makes the path recommendations for the various exercises when a user fits those classifications. In the past, three somatotypes were identified in morphology to classify body types based on different physical attributes: endomorph, mesomorph and ectomorph. However, the present disclosure in some examples expands those classifications to identify large numbers of classifications of users and sub user groups based on various attributes like limb length, DNA composition, and other Kinanthropometry related information about the user. By creating additional classifications beyond the three existing somatotypes, the disclosure is able to make better (e.g., more individualized) recommendations, as well as recommendations that may benefit related classifications of users. In this manner, the training assistant module 102 customizes the workouts to the particular user while optimizing to take advantage of any inherent strengths the user possesses, as well as address weaknesses, or other mobility concerns such as previous injuries that limit range of motion. Moreover, and for example, a deviation between corresponding time segments of the actual and expected path of motion may indicate a performance deficiency of the user's ability to perform the physical activity (e.g., the user is slowing down near the top end of a bench press to lock out the elbows, which may indicate a performance deficiency of the user's triceps (triceps brachii muscle)). In another example, during a bench press exercise the user may move the user's elbows in an incorrect angle due during the upwards press, which is detected to be caused by a weakness in the user's latissimus dorsi. If the user cannot raise the bar off the chest when lowered it may be caused by weak deltoids and/or weak pectoralis major.

Based on any identified deficiencies, the training assistant module 102 is configured to generate one or more suggestions that facilitate remediating the identified deviation(s). In some examples, the generated suggestion is a supplemental activity that facilitates remediating the identified deviation. In other words, the suggestions generated by the training assistant module 102 may indicate other exercises that facilitate fixing (e.g., improving) the user's weakness identified by the deviation(s). For example, if an identified deviation indicates that during a bench press the progress of the bar from the midpoint off the chest to lockout is slow, the generated suggestion may recommend a narrow bench press exercise and/or other tricep exercises to improve the strength of the triceps (e.g., push-ups, dips, skull crushers, dumbbell extensions, etc.). In another example, an identified deviation may indicate that the user is slowing down on an uphill portion of a run and the generated suggestion may recommend additional exercises that build quad strength (e.g., running backwards, doing squats and/or leg extensions, etc.). In yet another example, if the identified deviation indicates that the user is not squatting deep enough during a squatting exercise, the suggestion generated by the training assistant module 102 may recommend that the user performs supplemental box squat exercises and/or the device 100 can indicate a notification that will vibrate when the user hits depth (e.g., taking the weight down low enough in the exercise to properly perform the exercise). The depth can be computed by the device 100 from sensor data measuring deceleration or downward movement against time when the bar pauses and return to upward motion. Users can calibrate the system (e.g., devices, components, apparatuses, methods, operations, etc.) with a light weight on the bar and perform the full range of motion the user is capable of in the case of injury or simply wanting to train the system to the specific adaptations of the user. In some examples, the system takes into account the limited range of motion of the user due to injury, for example, and does not make recommendations for a full bar path while the user is recovering, or when the limited range of motion is deemed acceptable forever if the injury is permanent. Often, the range of motion deviates from the learned or reference model path the closer the user gets to performing the exercise with the additional load of near the maximum weight the user can lift when performing the exercise or when the user exceeds the weight, repetitions, and/or duration the user can handle when performing an exercise. The training assistant module 102 may track the long-term progress of the user, identify when it is recommended that the user changes the user's fitness routine, and suggest supplemental activities that keep the user's progress moving forward, in some examples. The body adapts to training stresses and users often hit plateaus in performance which may require changes to the training regimen to continue to achieve gains and results on the path to elite athletic performance results. Optionally, the suggestions generated by the training assistant module 102 are generated using the AI sub-module 116 described herein.

The electronic device 100 includes a user interface 114 that is communicatively coupled to the training assistant module 102 for receiving the suggestions generated by the training assistant module 102. The user interface 114 presents the generated suggestions to the user. The suggestions generated by the training assistant module 102 optionally are categorized as easy, medium, and hard when presented to the user. The easy suggestions may take a longer time of conditioning to achieve the correction in the performance of the exercise, while at the other end of the spectrum the hard suggestions prescribe a more intense but likely faster fix to the performance issues detected by the training assistant module 102. In view of the above, it should be understood that the electronic device 100 provides automatic physical training by tracking a process of exercising and suggesting improvements correlated with the exercise when weakness is automatically detected in the exercise.

The training assistant module 102 optionally includes the AI sub-module 116 that tracks, over time, the results of the generated suggestions as implemented by the user, crowdsources the tracked results to determine the effectiveness of the generated suggestion, and adjusts the generated suggestions for the user and/or other users based on the determined effectiveness. For example, the AI sub-module 116 is communicatively coupled to a cloud service 112 for receiving other users' results of implementing suggestions generated by other electronic devices (i.e., other automatic training assistant modules). The AI sub-module 116 collects the other user's results from the cloud service 112 and uses the other user's results in combination with the user's results from the training assistant module 102 to adjust the generated suggestions for the user and other users. The AI sub-module 116 may interact with information about the individual, such as, but not limited to, recovering from an injury, muscle strain, etc. In some examples, the individual may provide access to the individual's DNA information about the individual's fast or slow twitch muscle fiber composition so that the system can adjust the training regimen accordingly up or down in intensity, expand or narrow the number of assistance exercises, variety (e.g. a conjugate method), duration, and/or the like to achieve desired improvement. The results of many training assistant modules may be used such that the suggestions generated by the training assistant modules may be improved through crowdsourcing in this manner. In other words, the AI sub-module 116 may measure compliance with the generated suggestions (e.g., in view of age, genetics, diet, sleep habits, etc.) to infer feedback on how the suggestions worked to then learn and generate improved suggestions for the user and/or other users. In addition or alternatively to using the AI sub-module 116, crowdsourcing and adjusting the suggestions generated by training assistant modules may be performed by AI functionality at the cloud service 112. The system can even learn from users who perform ad-hoc exercises, diet and sleep and recovery information, and/or the like to build the most effective training regimen research.

In some examples, the AI sub-module 116 comprises a trained regressor such as, but not limited to, a random decision forest, directed acyclic graph, support vector machine, neural network, other trained regressor, and/or the like. Examples of trained regressors include a convolutional neural network and a random decision forest. It should further be understood that the AI sub-module 116, in some examples, may operate according to machine learning principles and/or techniques known in the art without departing from the systems and/or methods described herein.

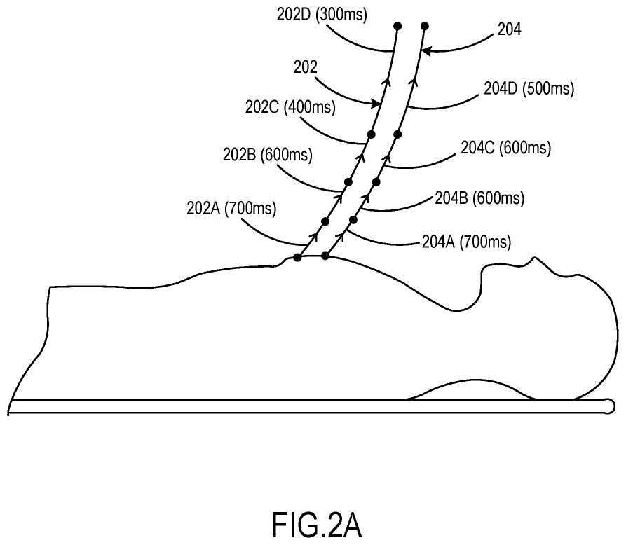

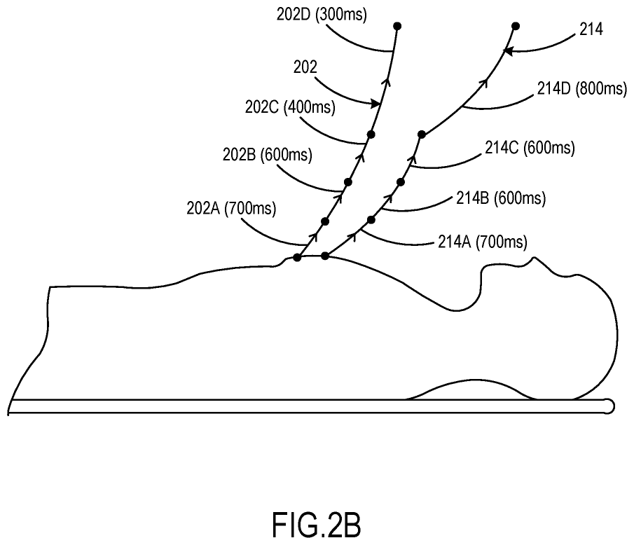

FIG. 2A is a diagram illustrating an exemplary bench press activity performed by the user. The activity model 104 (shown in FIG. 1) that models the bench press activity includes an expected or preferred path of motion 202 of the ascending phase of the bench press activity that corresponds to the user's physiology. As shown in FIG. 2A, the expected path of motion 202 is segmented into a plurality of time segments 202A, 202B, 202C, and 202D. FIG. 2A also illustrates the actual path of motion 204 (of the ascending phase) taken by the user while performing the bench press activity as recorded by the sensors 108 and/or 110 (shown in FIG. 1) of the electronic device 100 (shown in FIG. 1). The actual path of motion 204 is segmented into a plurality of time segments 204A, 204B, 204C, and 204D that correspond to the time segments 202A, 202B, 202C, and 202D, respectively, of the expected path of motion 202. Rather than being overlaid over the expected path of motion 202, the actual path of motion 204 is offset from the expected path of motion 202 in FIG. 2A for clarity.

In the example of FIG. 2A, the time segments 202A, 202B, 202C, and 202D of the expected path of motion 202 have respective time values of approximately 700 ms, 600 ms, 400 ms, and 300 ms, while the time segments 204A, 204B, 204C, and 204D of the actual path of motion 204 have time values of approximately 700 ms, 600 ms, 600 ms, and 500 ms, respectively. Accordingly, although the time segments 204A-D of the actual path of motion 204 are shown in FIG. 2A as having the same physical length as the corresponding time segments 202A-D of the expected path of motion 202, the example bench press activity of FIG. 2A illustrates a deviation of the actual path of motion 204 from the expected path of motion 202. For example, the time segments 204A and 204B of the actual path of motion 204 have approximately the same respective time values of approximately 700 ms and 600 ms as the corresponding time segments 202A and 202B of the expected path of motion 202. But, the time segment 204C of the actual path of motion 204 has an approximate time value of 600 ms, which deviates from the approximate time value of 400 ms of the corresponding time segment 202C of the expected path of motion 202. Moreover, in the example of FIG. 2A, the approximate time value of 500 ms of the time segment 204C of the actual path of motion 204 deviates from the approximate time value of 300 ms of the corresponding time segment 202D of the expected path of motion 202.

The example of FIG. 2A thus illustrates two deviations of the actual path of motion 204 from the expected path of motion 202 of the user's bench press activity. In such an example, as the greater time values of the time segments 204C and 204D occur near the middle and end of the path of motion of the bench press activity, the training assistant module 102 (shown in FIG. 1) may generate a suggestion that recommends a tricep exercise (e.g., a narrow-grip bench press) to improve the strength of the user's triceps. Based on the user's previous or subsequent performance with the accessory muscle training (e.g., triceps), the training assistant module 102 may choose a secondary or tertiary muscle group (besides triceps) to focus on as the highest priority weakness to improve first.

In the example of FIG. 2B, deviations are shown of the actual path of motion 214 from the same expected path of motion shown previously in path 202 A-D in FIG. 2A. The expected (or preferred) path 202 versus the actual path 214 with significant deviations in path is shown in FIG. 2B as parallel paths originating from different points on the chest with different time values for each similarly height segment with different lengths based on the bar path. Note the actual path 214 shown in FIG. 2B is illustrated with more sloped and less vertical path segments as compared to the expected path 202 and therefore has some longer length and longer time duration segments. For example, the time segments 214A and 214B of the actual path of motion 214 have approximately the same respective time values of approximately 700 ms and 600 ms as the corresponding time segments 202A and 202B of the expected path of motion 202. But, the time segment 214D of the actual path of motion 214 has an approximate time value of 800 ms, which deviates from the approximate time value of 300 ms of the corresponding time segment 202D of the expected path of motion 202. Some of the angles of the actual path 214 of the bar are different than the expected path 202 and therefore the segments' length and timing will change as the user performs the exercise out of the expected path 202. Some segments will be computed as same length but some segments may be longer in length and/or time based on the path the user takes to complete them as determined by the sensor data and computations that use the sensor information such as time, acceleration, direction of travel to compute the distance segments for the actual path 214 versus what was expected in the expected path 202. The system can identify the weaknesses that caused the bar path to deviate from the desired path from 202A-C and why the large inflection point where the actual path 214C to 214D was one of the major changes from expected path 202 to the actual path 214. Each of the segments is traceable back to one or more root causes of weakness and an appropriate remedy suggested by the training assistant module 102.

It should be appreciated that while FIG. 2A and FIG. 2B demonstrate examples of possibilities for variances in the actual path 214 from the expected path 202 in two dimensions (e.g., vertical height off the chest and horizontal as in left towards lower body and right towards head) for the bench press exercise, the bar in the user's hands does not always travel in a perfectly horizontal line. Thus, the sensors (on a wrist or in the bar) may detect a yaw or imbalance in the bar during the exercise and track that against the ideal bar path for level or evenness in the bar path in the upward direction. If the left or right arm raises up faster on one side this imbalance can be detected and a remedy to fix the imbalance in strength can be devised by the training assistant module 102. For example, the user may be left handed and therefore the left arm or left shoulder is more developed naturally through environmental conditioning so that the extra strength shows up when the user applies maximum effort when lifting weights. In this instance the training assistant module 102 would recommend additional isolation exercises for the right side to build up equal strength. However, some novice users may not know that doing three sets of right hand only overhead presses with a dumbbell could exhaust their tricep and/or shoulders before they bench press and they may have just unintentionally exhausted their muscles on the right side during this training session that shows up as a weakness during the bench press. The training assistance module 102 observes the user doing the pre-bench press workout exhausting repetitions on one arm and therefore it may not recommend additional isolation exercises to strengthen the right side because the wearable sensors will have recorded the activity that caused the temporary weakness that was detected in the bar path during the bench press. In this example, the training assistant module 102 may recommend as the remedy to rest and change the exercise order before the next time the user is recommended to perform the bench press exercise at maximum effort.

FIG. 3 illustrates a flow chart of a method 300 for automatic physical training using an electronic device according to an embodiment. The example method 300 is performed by an electronic device such as electronic device 100, and includes receiving, at 302, sensor data representing an actual path of motion of a user during a physical activity. At 304, the method 300 includes comparing the received sensor data to an identified activity model that includes an expected path of motion corresponding to the user's physiology. The method 300 further includes identifying, at 306, a deviation from the identified activity model (or from among plurality of acceptable models) based on the comparison. At 308, the method 300 includes generating a suggestion based on the identified deviation to remediate the identified deviation. The generated suggestion is presented, at 310, to the user.

FIG. 4 illustrates a flow chart of a method 400 for automatic physical training using an electronic device according to an embodiment. The example method 400 is performed by an electronic device such as electronic device 100, and includes receiving, at 402, sensor data representing an actual path of motion of a user during a physical activity. At 404, the method 400 includes comparing time segments of an actual path of motion of the received sensor data to time segments of an expected path of motion of an identified activity model. The expected path of motion corresponds to the user's physiology.

Optionally, the method 400 includes identifying, at 404A, the activity model by reading the received sensor data, comparing the sensor data to a plurality of activity models that represent different physical activities, and identifying the activity model based on the comparison. At 404B, the method 400 optionally includes at least one of constructing or adjusting the expected path of motion of at least one activity model by reading sensor data that represents a baseline path of motion of the user for the corresponding physical activity.

At 406, the method 400 includes identifying a deviation from the identified activity model based on the comparison. If it is determined at 406 that there are no deviations from the identified activity model, the method 400 includes taking, at 408, no further action. If a deviation is determined at 406, at 410 the method 400 includes generating, based on the identified deviation, a suggestion of a supplemental activity that facilitates remediating the identified deviation. At 412, the method 400 includes presenting the generated suggestion to the user.

At 414, the method 400 optionally includes tracking, over time, the results of the generated suggestion as implemented by the user, crowdsourcing the tracked results to determine the effectiveness of the generated suggestion, and adjusting the generated suggestion for other users based on the determined effectiveness.

In an example of the method 400, if a deviation identified at 406 indicates that during a bench press the progress of the bar from the midpoint off the chest to lockout is slow, a suggestion is generated at 410 that recommends a narrow bench press exercise and/or other tricep exercises to improve the strength of the user's triceps. In another example of the method 400, as a user repeats a squat movement and adds more weight, sensors detect the distance traveled by the user up and down by monitoring the acceleration and/or time of the user's movement along the path of motion. Alternatively, the system compares against the exercise model learned during a light or weight free range of motion training session. If a deviation determined at 406 indicates that the user is not squatting deep enough during, for example when heavier weights are used, a suggestion generated at 410 may recommend that the user performs supplemental box squat exercises, leg extensions, leg presses, hamstring, lower posterior chain exercises, target the adductors (e.g. adductor brevis, adductor longus, adductor magnus, adductor minimus, etc.), calf raises, etc.

In one example of the method 400, if a deviation identified at 406 indicates that the user is slowing down on an uphill portion of a run, the method 400 may include generating at 410 a suggestion that recommends additional exercises that build quad strength (e.g., running backwards), leg extensions, deep knee lunges, etc. In another example of the method 400, if a deviation of a time segment that occurs near the beginning of a strength exercise is identified at 406, the suggestion generated at 410 may recommend a fly exercise that exercises the user's pectoralis major muscles. Similarly, if a deviation of a time segment that occurs near the end of a strength exercise is identified at 406, the suggestion generated at 410 may recommend a forearm exercise.

In one example of the method 400, if a deviation identified at 406 indicates that the user is generating lower club head speeds while swinging a golf club, the suggestion generated at 410 may recommend an arm and/or shoulder exercise that increases the user's strength and thereby improves the club head speed of the user's golf swing. As a training aid, the system may recommend that the user performs faster repetitions of one or more sets of a physical activity to remediate a deviation. In some examples, the system may slowly recommend increased ranges of motion rather than risk injury to the athlete as the increases the range of motion under load or while training, so the user can remain injury free as long as possible. The system may recommend stretching exercises to improve range of motion, such as using bands for a hip flexor stretch to “open the hip flexors” or using a kneeling hip flexor stretch, as examples from a plurality of possibilities, which are known to help some athletes improve squatting technique and performance.

### Additional Examples

In one example scenario, the methods, systems, and electronic devices described herein may be used to track the long term progress of the user, identify when it is recommended that the user changes the user's fitness routine, and suggest supplemental activities that keep the user's progress moving forward, in some examples.

In another example scenario, the methods, systems, and electronic devices described herein may provide a training functionality wherein a baseline path of motion of the user is established for the physical activity and the user's performance is then tracked over repeated performances of the physical activity. For example, the user may perform a bench press with no weight on the bar bell to establish a baseline bench press path of motion. As the user progressively adds more weight to the bar bell, the user's actual path of motion is tracked until deviations from the baseline path of motion are identified. Another example of a training mode functionality includes learning a user's training limitations (e.g., a suggestion is generated for a user to only perform a half-rep bench press because of a previous shoulder injury).

### Exemplary Operating Environment

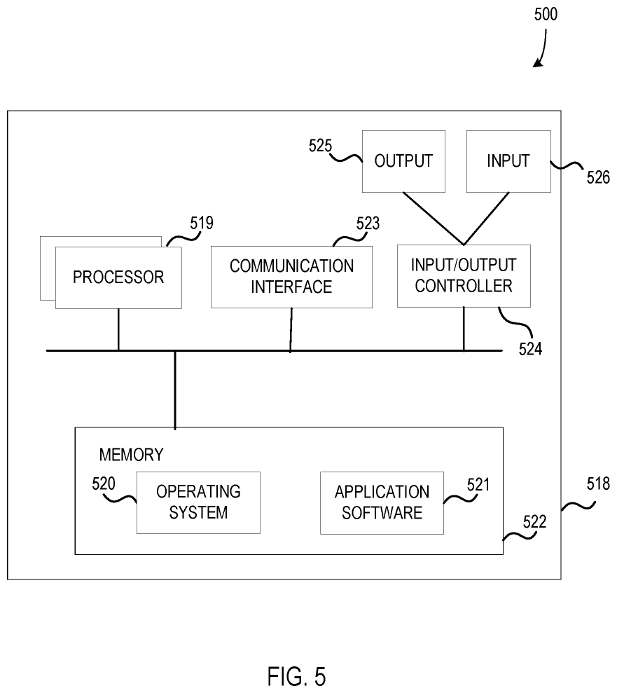

The present disclosure is operable with an electronic device (i.e., a computing apparatus) according to an embodiment as a functional block diagram 500 in FIG. 5. In an embodiment, components of a computing apparatus 518 may be implemented as a part of an electronic device according to one or more embodiments described in this specification. The computing apparatus 518 comprises one or more processors 519 which may be microprocessors, controllers or any other suitable type of processors for processing computer executable instructions to control the operation of the electronic device. Platform software comprising an operating system 520 or any other suitable platform software may be provided on the apparatus 518 to enable application software 521 to be executed on the device.

Computer executable instructions may be provided using any computer-readable media that are accessible by the computing apparatus 518. Computer-readable media may include, for example, computer storage media such as a memory 522 and communications media. Computer storage media, such as a memory 522, include volatile and non-volatile, removable and non-removable media implemented in any method or technology for storage of information such as computer readable instructions, data structures, program modules or the like. Computer storage media include, but are not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing apparatus. In contrast, communication media may embody computer readable instructions, data structures, program modules, or the like in a modulated data signal, such as a carrier wave, or other transport mechanism. As defined herein, computer storage media do not include communication media. Therefore, a computer storage medium should not be interpreted to be a propagating signal per se. Propagated signals per se are not examples of computer storage media. Although the computer storage medium (the memory 522) is shown within the computing apparatus 518, it will be appreciated by a person skilled in the art, that the storage may be distributed or located remotely and accessed via a network or other communication link (e.g. using a communication interface 523).

The computing apparatus 518 may comprise an input/output controller 524 configured to output information to one or more output devices 525, for example a display or a speaker, which may be separate from or integral to the electronic device. The input/output controller 524 may also be configured to receive and process an input from one or more input devices 526, for example, a keyboard, a microphone or a touchpad. In one embodiment, the output device 525 may also act as the input device. An example of such a device may be a touch sensitive display. The input/output controller 524 may also output data to devices other than the output device, e.g. a locally connected printing device. In some embodiments, a user 527 may provide input to the input device(s) 526 and/or receive output from the output device(s) 525.

The functionality described herein can be performed, at least in part, by one or more hardware logic components. According to an embodiment, the computing apparatus 518 is configured by the program code when executed by the processor 519 to execute the embodiments of the operations and functionality described. Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Program-specific Standard Products (ASSPs), System-on-a-chip systems (SOCs), Complex Programmable Logic Devices (CPLDs), Graphics Processing Units (GPUs).

Although some of the present embodiments may be described and illustrated as being implemented in a smartphone, a mobile phone, a wearable device, or a tablet computer, these are only examples of a device and not a limitation. As those skilled in the art will appreciate, the present embodiments are suitable for application in a variety of different types of devices, such as portable and mobile devices, for example, in laptop computers, tablet computers, game consoles or game controllers, various wearable devices, etc.

At least a portion of the functionality of the various elements in the figures may be performed by other elements in the figures, or an entity (e.g., processor, web service, server, application program, computing device, etc.) not shown in the figures.

Although described in connection with an exemplary computing system environment, examples of the disclosure are capable of implementation with numerous other general purpose or special purpose computing system environments, configurations, or devices.

Examples of well-known computing systems, environments, and/or configurations that may be suitable for use with aspects of the disclosure include, but are not limited to, mobile computing devices, personal computers, server computers, hand-held or laptop devices, multiprocessor systems, gaming consoles, microprocessor-based systems, set top boxes, smart televisions, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein.

In examples involving a general-purpose computer, aspects of the disclosure transform the general-purpose computer into a special-purpose computing device when configured to execute the instructions described herein.

The examples illustrated and described herein as well as examples not specifically described herein but within the scope of aspects of the disclosure constitute exemplary means for automatic physical training using an electronic device. For example, the elements illustrated in FIG. 1, such as when encoded to perform the operations illustrated in FIGS. 3 and 4, constitute exemplary means for automatic physical training using an electronic device.

Alternatively or in addition to the other examples described herein, examples include any combination of the following:
An electronic device comprising:
at least one processor;
at least one memory storing activity models that include expected paths of motion corresponding to a user's physiology;
a training assistant module that, in response to execution by the at least one processor, receives sensor data representing an actual path of motion of a user during a physical activity and compares the received sensor data to an identified one of the activity models, the training assistant module, in response to execution by the at least one processor, identifies a deviation from the identified activity model based on the comparison and generates a suggestion based on the identified deviation to remediate the identified deviation; and
a user interface that, in response to execution by the at least one processor, presents the suggestion generated by the training assistant module to the user.
wherein the training assistant module, in response to execution by the processor, generates the suggestion as a supplemental activity that facilitates remediating the identified deviation.
wherein the training assistant module comprises an artificial intelligence (AI) sub-module that, in response to execution by the processor, tracks, over time, the results of the generated suggestion as implemented by the user and crowdsources the tracked results to determine the effectiveness of the generated suggestion, the AI sub-module, in response to execution by the processor, adjusts the generated suggestion for other users based on the determined effectiveness.
wherein the expected paths of motion of the activity models stored by the memory are segmented into time segments, the training assistant module, upon execution by the processor, compares time segments of the actual path of motion of the received sensor data to the time segments of the expected path of motion of the identified activity model to identify the identified deviation.
wherein the training assistant module, upon execution by the processor, at least one of constructs or adjusts the expected path of motion of at least one of the activity models by reading sensor data that represents a baseline path of motion of the user for the corresponding physical activity.
wherein the training assistant module, in response to execution by the processor, reads the sensor data and compares the sensor data to the activity models stored by the memory to identify the identified activity model based on the comparison.
wherein the training assistant module comprises an artificial intelligence (AI) sub-module that, in response to execution by the processor, generates the suggestion based on the identified deviation.
wherein the electronic device is a wearable device.
A computerized method comprising:
receiving, at an electronic device, sensor data representing an actual path of motion of a user during a physical activity;
comparing the received sensor data to an identified activity model that includes an expected path of motion corresponding to the user's physiology;
identifying a deviation from the identified activity model based on the comparison;
generating a suggestion based on the identified deviation to remediate the identified deviation; and
presenting the generated suggestion to the user.
wherein generating the suggestion includes generating a supplemental activity that facilitates remediating the identified deviation.
further comprising tracking, over time, the results of the generated suggestion as implemented by the user, crowdsourcing the tracked results to determine the effectiveness of the generated suggestion, and adjusting the generated suggestion for other users based on the determined effectiveness.
wherein comparing the received sensor data to the identified activity model comprises comparing time segments of the actual path of motion of the received sensor data to time segments of the expected path of motion of the identified activity model.
further comprising at least one of constructing or adjusting the expected path of motion of at least one activity model by reading sensor data that represents a baseline path of motion of the user for the corresponding physical activity.
further comprising reading the received sensor data, comparing the sensor data to a plurality of activity models that represent different physical activities, and identifying the identified activity model based on the comparison.
One or more computer storage media having computer-executable instructions that, in response to execution by a processor, cause the processor to at least:
receive sensor data representing an actual path of motion of a user during a physical activity;
compare the received sensor data to an identified activity model that includes an expected path of motion corresponding to the user's physiology;
identify a deviation from the identified activity model based on the comparison;
generate a suggestion based on the identified deviation to remediate the identified deviation; and
present the generated suggestion to the user.
wherein the processor generates the suggestion as a supplemental activity that facilitates remediating the identified deviation.
wherein the processor is further caused to track, over time, the results of the generated suggestion as implemented by the user, crowdsource the tracked results to determine the effectiveness of the generated suggestion, and adjust the generated suggestion for other users based on the determined effectiveness.
wherein the processor is caused to compare the received sensor data to the identified activity model by comparing time segments of the actual path of motion of the received sensor data to time segments of the expected path of motion of the identified activity model.
wherein the processor is further caused to at least one of construct or adjust the expected path of motion of at least one activity model by reading sensor data that represents a baseline path of motion of the user for the corresponding physical activity.
wherein the processor is further caused to read the received sensor data, compare the sensor data to a plurality of activity models that represent different physical activities, and identify the identified activity model based on the comparison.

While no personally identifiable information is tracked by aspects of the disclosure, examples have been described with reference to data monitored and/or collected from the users. In some examples, notice may be provided to the users of the collection of the data (e.g., via a dialog box or preference setting) and users are given the opportunity to give or deny consent for the monitoring and/or collection. The consent may take the form of opt-in consent or opt-out consent.

Any range or device value given herein may be extended or altered without losing the effect sought, as will be apparent to the skilled person.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

The term “comprising” is used in this specification to mean including the feature(s) or act(s) followed thereafter, without excluding the presence of one or more additional features or acts.

In some examples, the operations illustrated in the figures may be implemented as software instructions encoded on a computer readable medium, in hardware programmed or designed to perform the operations, or both. For example, aspects of the disclosure may be implemented as a system on a chip or other circuitry including a plurality of interconnected, electrically conductive elements.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. An electronic device comprising:
at least one processor;
at least one memory storing activity models that define expected paths of motion corresponding to physiology of a user during physical activities;
a training assistant module that, in response to execution by the at least one processor, receives sensor data representing an actual path of motion of the user during one of the physical activities and compares the received sensor data to an identified one of the activity models, the training assistant module, in response to execution by the at least one processor, identifies a deviation from the identified activity model based on the comparison and generates a suggestion based on the identified deviation to remediate the identified deviation; and
a user interface that, in response to execution by the at least one processor, presents the suggestion generated by the training assistant module to the user.

2. The electronic device of claim 1, wherein the training assistant module, in response to execution by the processor, generates the suggestion as a supplemental activity that facilitates remediating the identified deviation.

3. The electronic device of claim 1, wherein the training assistant module comprises an artificial intelligence (AI) sub-module that, in response to execution by the processor, tracks, over time, the results of the generated suggestion as implemented by the user and crowdsources the tracked results to determine the effectiveness of the generated suggestion in remediating the identified deviation, the AI sub-module, in response to execution by the processor, adjusts the generated suggestion for other users based on the determined effectiveness.

4. The electronic device of claim 1, wherein the expected paths of motion of the activity models stored by the memory are segmented into time segments, the training assistant module, upon execution by the processor, compares time segments of the actual path of motion of the received sensor data to the time segments of the expected path of motion of the identified one of the activity models to identify the identified deviation.

5. The electronic device of claim 1, wherein the training assistant module, upon execution by the processor, at least one of constructs or adjusts the expected path of motion of at least one of the activity models by reading sensor data that represents a baseline path of motion of the user for a physical activity corresponding to the at least one of the activity models.

6. The electronic device of claim 1, wherein the training assistant module, in response to execution by the processor, reads the sensor data and compares the sensor data to the activity models stored by the memory to identify the identified one of the activity models based on the comparison.

7. The electronic device of claim 1, wherein the training assistant module comprises an artificial intelligence (AI) sub-module that, in response to execution by the processor, generates the suggestion based on the identified deviation.

8. The electronic device of claim 1, wherein the electronic device is a wearable device.

9. A computerized method comprising:
receiving, at an electronic device, sensor data representing an actual path of motion of a user during a physical activity;
comparing the received sensor data to an activity model that includes an expected path of motion corresponding to a physiology of the user;
identifying a deviation from the activity model based on the comparison;
generating a suggestion based on the identified deviation to remediate the identified deviation; and
presenting the generated suggestion to the user.

10. The computerized method of claim 9, wherein generating the suggestion includes generating a supplemental activity that facilitates remediating the identified deviation.

11. The computerized method of claim 9, further comprising tracking, over time, the results of the generated suggestion as implemented by the user, crowdsourcing the tracked results to determine the effectiveness of the generated suggestion in remediating the identified deviation, and adjusting the generated suggestion for other users based on the determined effectiveness.

12. The computerized method of claim 9, wherein comparing the received sensor data to the activity model comprises comparing time segments of the actual path of motion of the received sensor data to time segments of the expected path of motion of the identified activity model.

13. The computerized method of claim 9, further comprising at least one of constructing or adjusting the expected path of motion of at least one activity model by reading sensor data that represents a baseline path of motion of the user for the physical activity corresponding to the at least one activity model.

14. The computerized method of claim 9, further comprising reading the received sensor data, comparing the sensor data to a plurality of activity models that represent different physical activities, and identifying the activity model from the plurality of activity models based on the comparison.

15. One or more computer storage media having computer-executable instructions that, in response to execution by a processor, cause the processor to at least:
receive sensor data representing an actual path of motion of a user during a physical activity;
compare the received sensor data to an activity model that includes an expected path of motion corresponding to a physiology of the user;
identify a deviation from the activity model based on the comparison;
generate a suggestion based on the identified deviation to remediate the identified deviation; and
present the generated suggestion to the user.

16. The one or more computer storage media of claim 15, wherein the processor generates the suggestion as a supplemental activity that facilitates remediating the identified deviation.

17. The one or more computer storage media of claim 15, wherein the processor is further caused to track, over time, the results of the generated suggestion as implemented by the user, crowdsource the tracked results to determine the effectiveness of the generated suggestion in remediating the identified deviation, and adjust the generated suggestion for other users based on the determined effectiveness.

18. The one or more computer storage media of claim 15, wherein the processor is caused to compare the received sensor data to the activity model by comparing time segments of the actual path of motion of the received sensor data to time segments of the expected path of motion of the activity model.

19. The one or more computer storage media of claim 15, wherein the processor is further caused to at least one of construct or adjust the expected path of motion of at least one activity model by reading sensor data that represents a baseline path of motion of the user for the physical activity corresponding to the at least one activity model.

20. The one or more computer storage media of claim 15, wherein the processor is further caused to read the received sensor data, compare the sensor data to a plurality of activity models that represent different physical activities, and identify the activity model based on the comparison.
